(19) United States
(12) Patent Application Publication
(10) Pub. No.: US 2018/0280185 A1
(43) Pub. Date: Oct. 4, 2018

(54) SPLINT

(71) Applicant: NEO G LIMITED, Yorkshire (GB)

(72) Inventors: Paul Starkey, Yorkshire (GB); Okan Ozturkatalay, Istanbul (TR)

(21) Appl. No.: 15/765,288

(22) PCT Filed: Sep. 29, 2016
(86) PCT No.: PCT/GB2016/053040

§ 371 Date: Apr. 2, 2018

(30) Foreign Application Priority Data
Oct. 2, 2015 (GB) 1517432.9

(51) Int. Cl. A61F 5/058

(52) U.S. Cl. CPC A61F 5/05875

(57) ABSTRACT

A digit immobilizing splint is provided. The splint includes a first support portion operable in use to abut and support an underneath face of a digit on a first side of a joint of the digit, a second support portion operable in use to abut and support the underneath face of the digit on a second side of the joint, first and second fixing portions operable in use to respectively abut opposing side faces of the digit. The first and second fixing portions each extend between the first support portion and the second support portion. The first and second fixing portions are operable to be moved between a first, open, position wherein the first and second fixing portions do not abut the digit when the first and second support portions are abutting the digit, and a second, closed, position wherein the first and second fixing portions abut opposing sides of the digit such as to immobilize the digit and hold the splint in position around the digit.

The present invention relates to a splint. More specifically, the present invention relates to a digit immobilising splint for a finger.

Splints are devices that immobilise body parts such as a limb, a digit or the spine. The splint may be used to immobilise the body part by restricting the movement of a broken bone and/or a damaged joint.

Splint for digits, for example for fingers, are attached along the longitudinal axis of the finger in order to restrict lateral movement of the digit beyond the metacarpophalangeal joint and/or to restrict flexion and extension about the proximal inter-phalangeal and/or the distal inter-phalangeal joints.

Many known splints use a fastening system in which the splint is wrapped around the finger and then secured by fastening the splint together, for example by using a clip on the upper face of the finger. However, finger splints may need to be applied by the user to one hand whilst using the other hand and it has been found that such fastening devices can be difficult to operate with one hand. As such, it is desirable to provide an easy to apply splint. In some instances, it may also be advantageous to remove the splint for a period to allow some unrestricted movement of the digit in order to aid recovery, as such it is also desirable to provide a splint that can easily be removed and re-used.

An example of a known splint is a splint that consists of a stiff board that is placed under the finger with bandages or tape used to secure the board in place by wrapping the bandage or tape around the finger and board. As well as being difficult to apply with one hand, such an arrangement can restrict airflow around the surface of the skin of the digit. As such, it would also be advantageous to provide a splint that improves aeration of the digit whilst the splint is in place.

It is therefore an object of aspects of the present invention to address one or more of the abovementioned or other problems. In particular, it is an object of the present invention to provide an easy to apply splint, more particularly an easy to apply splint that provides improved aeration of the immobilised digit.

According to a first aspect of the present invention there is provided a digit immobilising splint comprising:

a first support portion operable in use to abut and support an underneath face of a digit on a first side of a joint of the digit,

a second support portion operable in use to abut and support the underneath face of the digit on a second side of the joint,

first and second fixing portions operable in use to respectively abut opposing side faces of the digit,

wherein the first and second fixing portions each extend between the first support portion and the second support portion,

wherein the first and second fixing portions are operable to be moved between a first, open, position wherein the first and second fixing portions do not abut the digit when the first and second support portions are abutting the digit, and a second, closed, position wherein the first and second fixing portions abut opposing sides of the digit such as to immobilise the digit and hold the splint in position around the digit.

The splint of the present invention is operable to be held around the digit in the closed position without the presence of additional fastening means. For example, the splint does not require the use of fastening means extending over the upper face of the digit linking the first fixing portion to the second fixing portion in order to hold the splint in position. Suitably, in the closed position the fixing portions are connected only by connecting members extending over one of the underneath or the upper face of the digit, suitably the connecting members are the support portions. By “underneath face” it is meant the inner face of the digit during flexion of the digit.

Advantageously, a splint according to the present invention can be arranged on the digit by applying pressure to only one point on either side of the splint. Accordingly, the splint is easy and quick to apply. It can be applied by the user to their own digit using only two fingers of one hand. The first and second fixing means can be brought toward one another in one motion to fix the splint into place around the digit.

The splint may be used to immobilise a digit such as a finger, thumb or a toe, preferably a human finger.

The splint may comprise one or more further support portions operable to abut a further point on the underneath face of the digit.

The first, second and further support portions (when present) may be spaced apart. Suitably, the first, second and further support portions are longitudinally spaced apart by an aperture.

Suitably, the spaced support portions are connected together via the fixing portions. As such, structural rigidity between the support portions may be provided by the fixing portions.

The first and second fixing portions may each extend between the first support portion and the second support portion such as to form a loop, suitably a continuous loop. Preferably, the loop comprises an inner aperture extending through its vertical axis. Suitably, two opposed points of the loop are the first and second support portions, respectively, and sections of the loop connecting the support portions, and which can abut the side walls of the digit in use, are the first and second fixing portions.

Further support portions may extend from one fixing portion to the other across the aperture of the loop, suitably by extending across substantially the middle of the aperture.

Advantageously, as the fixing portions are in contact with the side faces of the digit, linking the spaced support portions via the fixing portions means that sections of the underneath face of the immobilised part of the digit remain uncovered, allowing improved aeration. Further, as no additional fastening means is required across the upper face of the digit between the fixing means, this section of the upper face of the immobilised part of the digit also remains uncovered. As such, a splint according to the present invention can provide significantly improved aeration in addition to ease of application.

One or more of the support portions may comprise a tab. Suitably the tab extends longitudinally outwardly from an end of the splint to provide an enlarged support portion relative to the other support portions. Preferably, the distal supporting portion comprises a tab.

The fixing portions may be operable to abut the upper face of the digit in addition to a side face of the digit. Suitably, the fixing portion is flexible such that its inner face is operable to be bent about the longitudinal axis of the splint to abut a side face of the digit and the upper face of the digit.

Suitably, the connection between the support and fixing portions is flexible to allow the fixing portions to be bent from the open to the closed position with manual pressure applied by the user. Preferably, the fixing portions may be bent from the open to the closed position without causing significant movement or bending of the support portions. Preferably, substantially the entire splint is flexible about the splint's lateral and longitudinal axis whilst in the open position.

Suitably, in order to hold the splint in position around the finger the flexible splint has a degree of stiffness that is sufficient to prevent the splint from reverting to the open position whilst in the closed position without the application of manual pressure by the user to the fixing portions.

When in the open position, the splint may be easily bendable about the longitudinal axis by the user. When in the open position, the splint may be easily bendable about the longitudinal and lateral axis by the user. Suitably, in the open position the splint is not easily bendable about the vertical axis by the user. Preferably, when the splint is in the closed position, the splint is not easily bendable about the lateral axis

Suitably, the fixing portions of the splint have a width that is greater than their depth when in the open position and a depth that is greater than their width when in the closed position.

Preferably, the fixing portions have a width of between 3 to 20 mm and a depth of 0.2 to 5 mm when in the open position and substantially reversed width and depth values when in the closed position. Suitably, the width values are between 3 to 16 mm, such as between 4 to 12 mm, 5 to 10 mm or 6 to 8 mm. Suitably the depth values are between 0.2 to 3 mm, such as 0.5 to 2.5 mm, 0.75 to 2 mm or 0.8 to 1.5 mm.

The splint may have a total width in the open position of between 30 mm and 150 mm, such as between 40 mm and 120 mm, or between 50 mm and 100, preferably between 55 mm and 95 mm.

Suitably the distance between the outer edges of the first and second support portions is between 30 mm and 150 mm, such as between 40 mm and 120 mm, or between 50 mm and 100, preferably between 55 mm and 95 mm.

In one embodiment, the splint has a first end that is wider than a second opposed end. Suitably, the first end and the second end are the longitudinal ends of the splint. Suitably, an end of the splint operable to be proximal to the base of the digit is wider than the end of the splint operable to be distal to the base of the digit.

Digits generally contain parts of varying diameter. A digit may taper from a large diameter to a smaller diameter. For example, the part of the finger distal from the proximal inter-phalangeal joint is generally narrower than the proximal inter-phalangeal joint. Advantageously, the varying widths of the ends of the splint allow the splint to accommodate the varying diameter of the digit. For instance, where the diameter of the digit is larger, the end of the splint having a larger width can be used to ensure that the fixing portions extend high enough up the side face of that part of the digit as to provide a strong hold around the digit. Likewise, providing an end with reduced width reduces the amount of material required in the splint and therefore lowers the cost.

Preferably, the splint has a planar conformation when in the open position. Advantageously, this provides a splint that may easily be transported, for example in a user's pocket and which may also easily be stacked for improved storage efficiency.

Preferably, the splint comprises the same components in the closed position as in the open position. As such, preferably the splint is operable to function as a single component.

The splint may be a laminate, such as a two, three, four, five, six or seven layer laminate. The splint may comprise a flexible core layer having the suitable level of flexibility and stiffness discussed above. Preferably, the core is suitably flexible and stiff thought out its body.

The core may be formed of metal or plastic. Preferably, the core is formed of metal, most preferably aluminium.

The splint may also comprise a padding layer operable to engage the surface of the digit. Suitably, the padding is a resiliently deformable material, such as a resiliently deformable foam. Preferably, the padding comprises neoprene or “viscoelastic” polyurethane foam/low-resilience polyurethane foam. The padding may be arranged on the support portions and the fixing portions. Suitably the padding is continuous around the loop of the splint.

The splint may comprise an upper layer, suitably a transparent upper layer. The upper layer may be formed of polyurethane.

The splint may comprise a first core layer and a second padding layer. Preferably, the padding layer is arranged on a face of the core layer.

The padding layer may have a greater width than the core layer. Suitably, the padding layer may have a width that is between 0.5-5 mm wider than the core layer, such as between 1-4 mm or 1-3 mm.

According to a further aspect of the present invention, there is provided the use of a splint according to the first aspect of the invention to immobilise a digit, suitably a finger, thumb or a toe.

According to a further aspect of the present invention, there is provided a method of immobilising a digit with a splint according to the first aspect of the present invention, comprising the steps of:
a. arranging the first and second support portions adjacent to, preferably in contact with, the underside face of the digit;
b. moving the first and second fixing portions from the open position to the closed position wherein the fixing portions abut opposing sides of the digit such as to immobilise the digit and hold the split in place around the digit.

Preferably, the splint is operable to be moved from the open position into the closed position with the application of pressure to a single point on each side of the splint, suitably a single point on each of the first and second fixing portions.

As such, the splint may be moved to the closed position with the use of only two fingers, and in one motion. Advantageously, the splint may therefore be quickly and easily applied.

According to a further aspect of the present invention, there is provided a computer-readable medium having computer-executable instructions adapted to cause a 3D printer to print a splint according to a first aspect of the present invention.

All of the features contained herein may be combined with any of the above aspects in any combination.

For a better understanding of the invention, and to show how embodiments of the same may be carried into effect, reference will now be made, by way of example only, to the following figures:

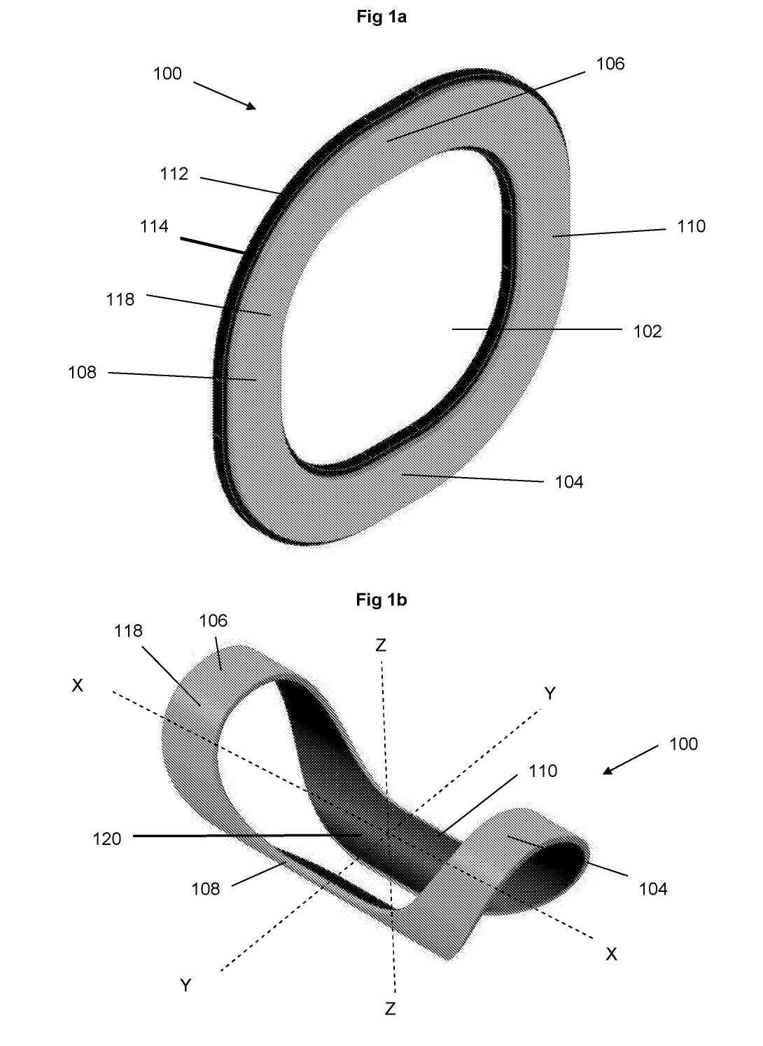

FIGS. 1a and 1b show perspective views of a first embodiment of a splint according to the first aspect of the present invention.

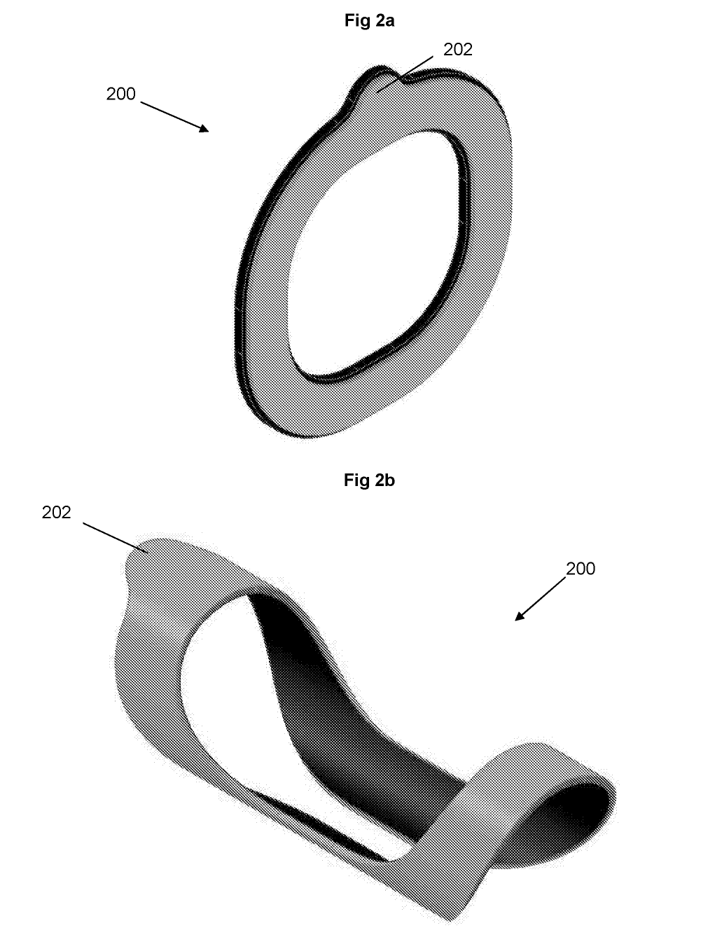

FIGS. 2a and 2b show perspective views of a second embodiment of a splint according to the first aspect of the present invention.

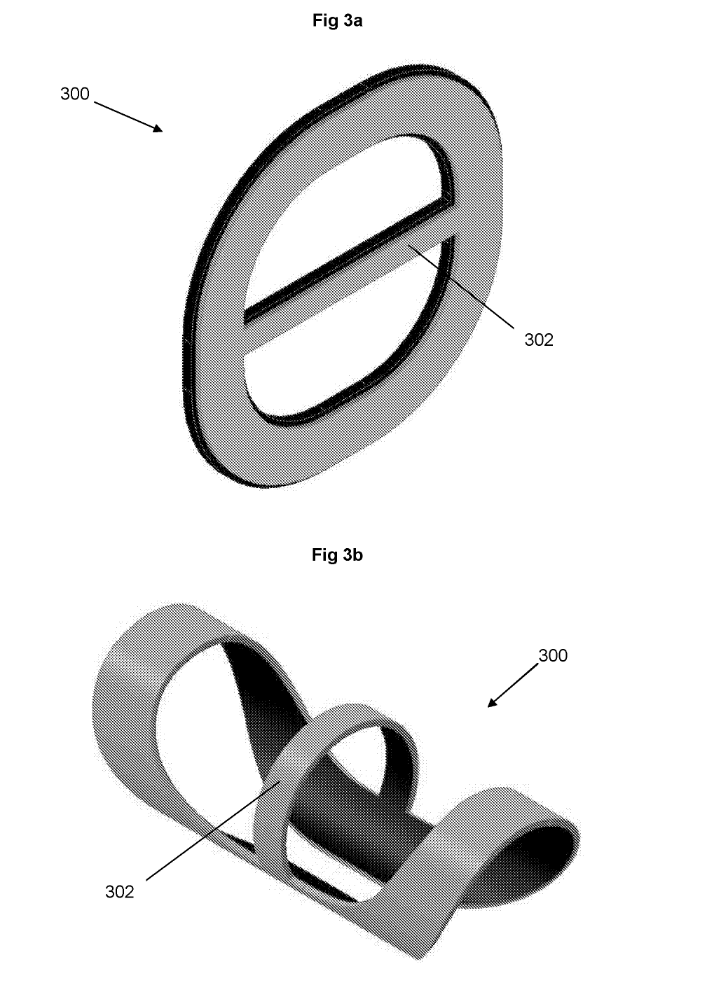

FIGS. 3a and 3b show perspective views of a third embodiment of a splint according to the first aspect of the present invention.

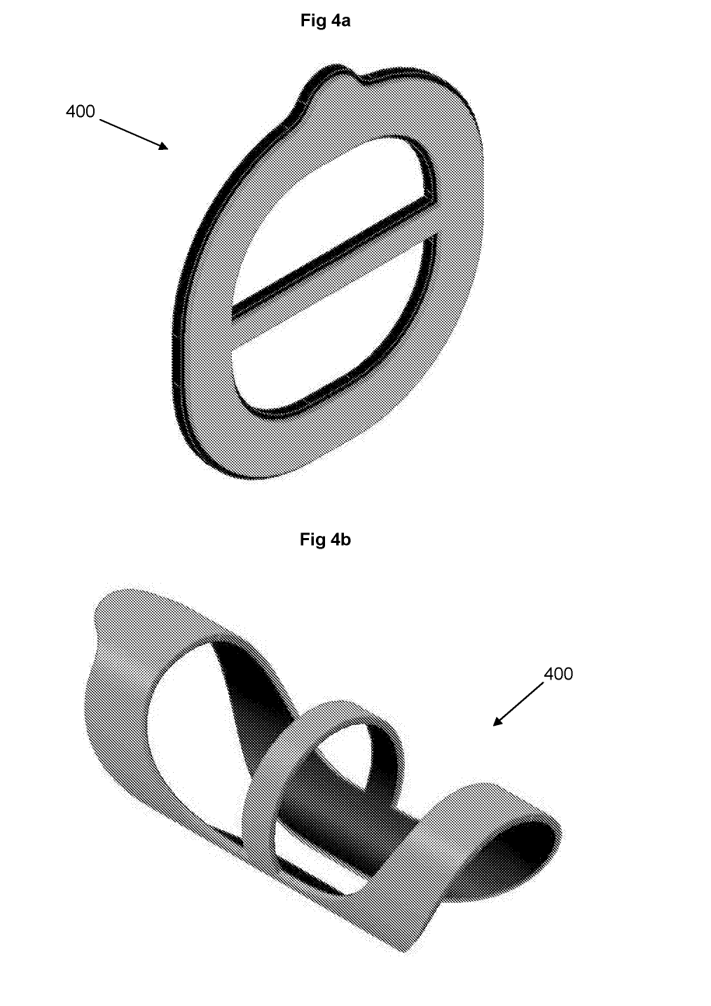

FIGS. 4a and 4b show perspective views of a fourth embodiment of a splint according to the first aspect of the present invention.

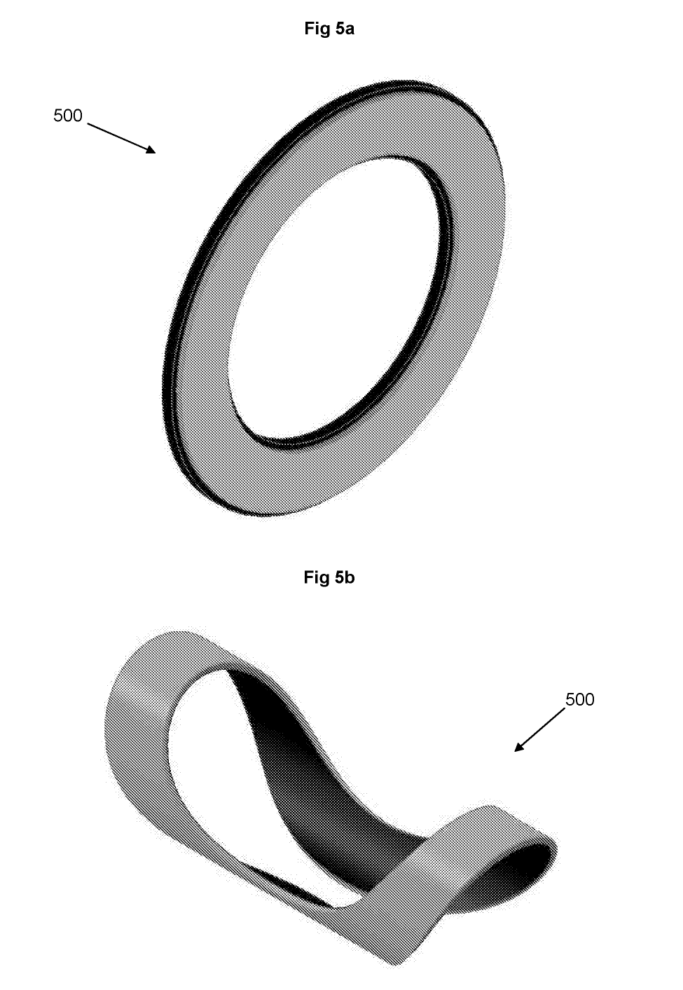

FIGS. 5a and 5b show perspective views of a fifth embodiment of a splint according to the first aspect of the present invention.

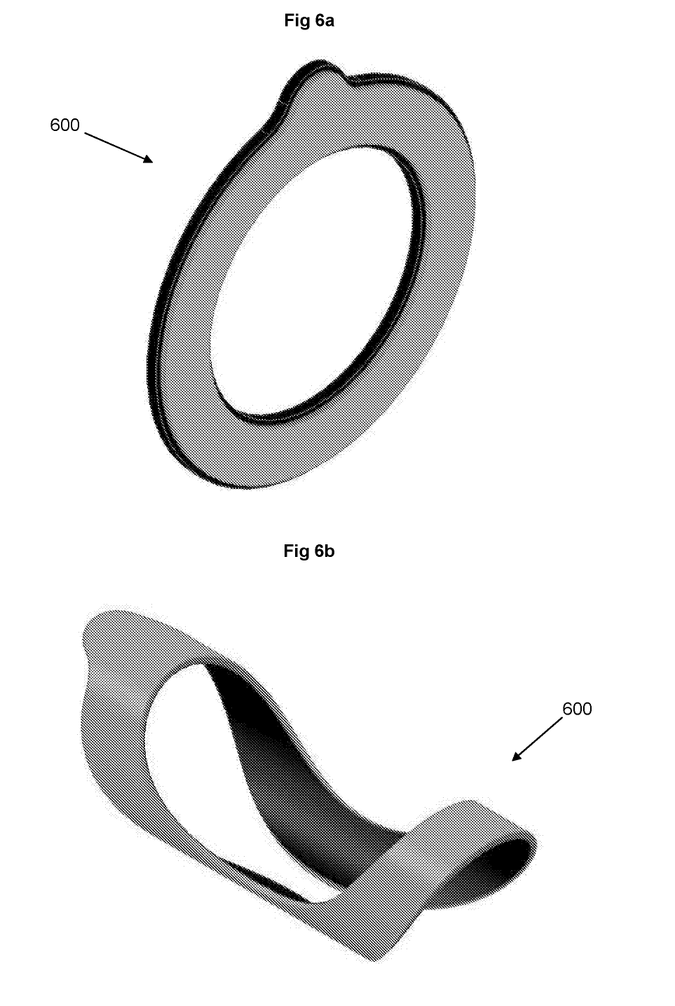

FIGS. 6a and 6b show perspective views of a sixth embodiment of a splint according to the first aspect of the present invention.

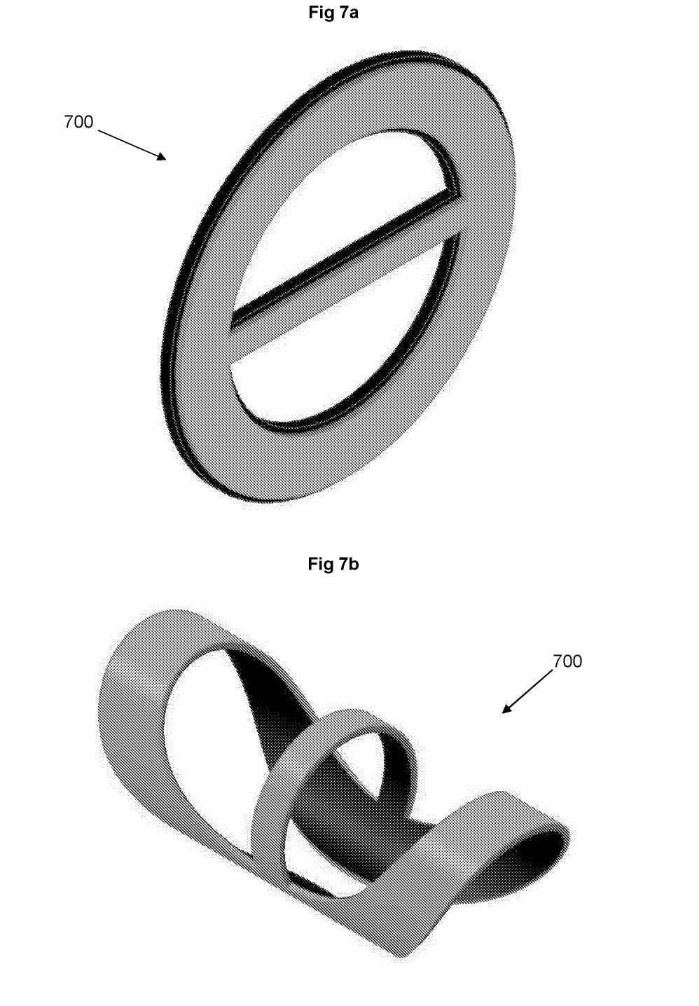

FIGS. 7a and 7b show perspective views of a seventh embodiment of a splint according to the first aspect of the present invention.

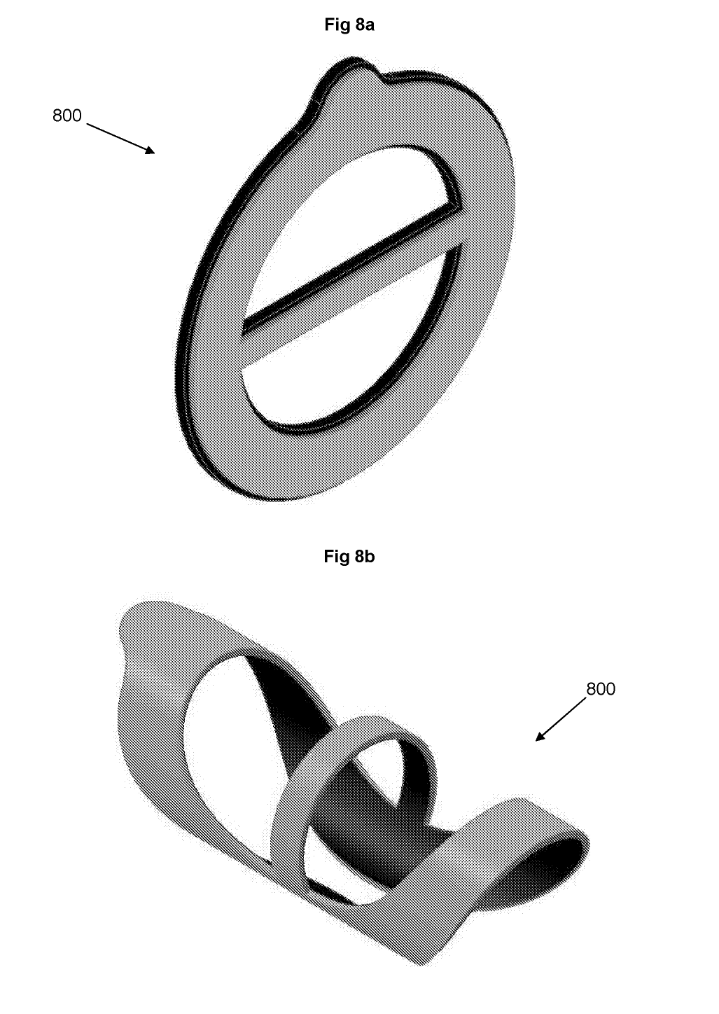

FIGS. 8a and 8b show perspective views of an eight embodiment of a splint according to the first aspect of the present invention.

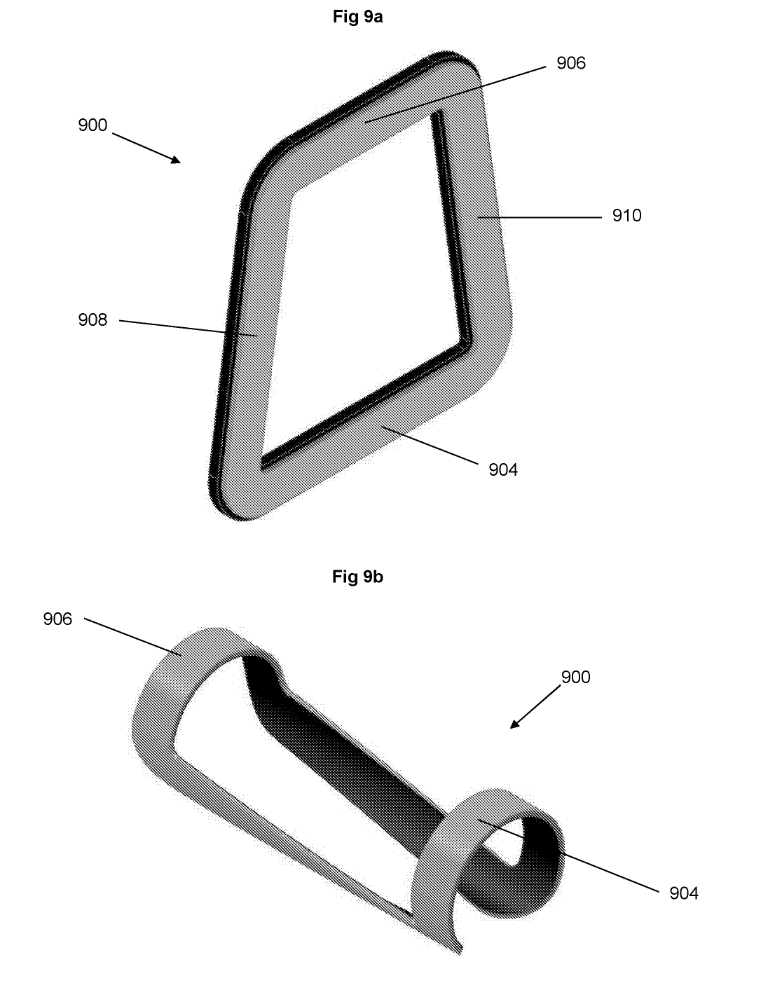

FIGS. 9a and 9b show perspective views of a ninth embodiment of a splint according to the first aspect of the present invention.

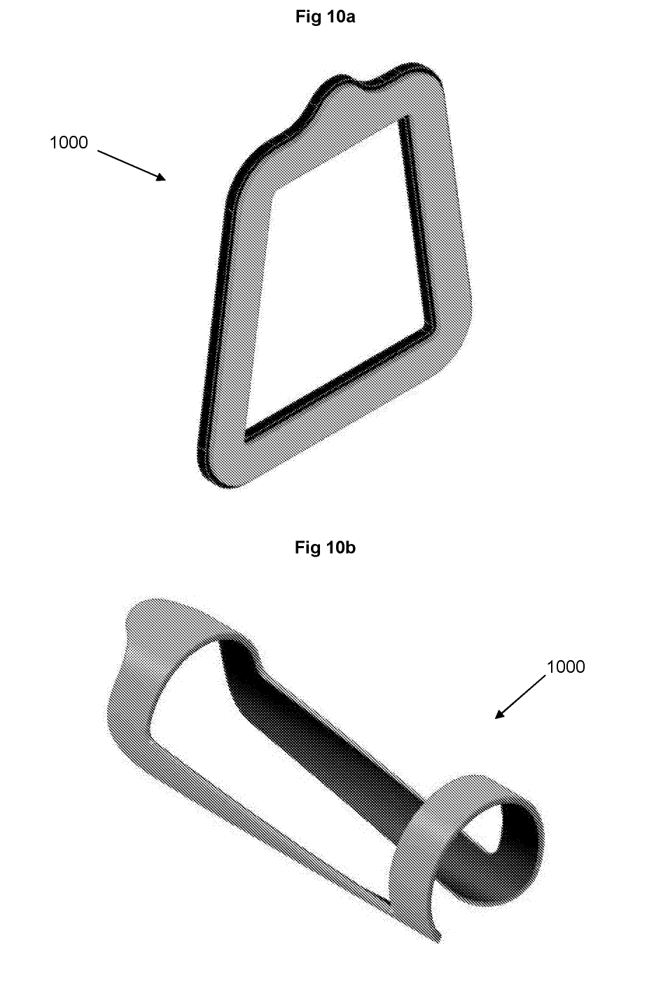

FIGS. 10a and 10b show perspective views of a tenth embodiment of a splint according to the first aspect of the present invention.

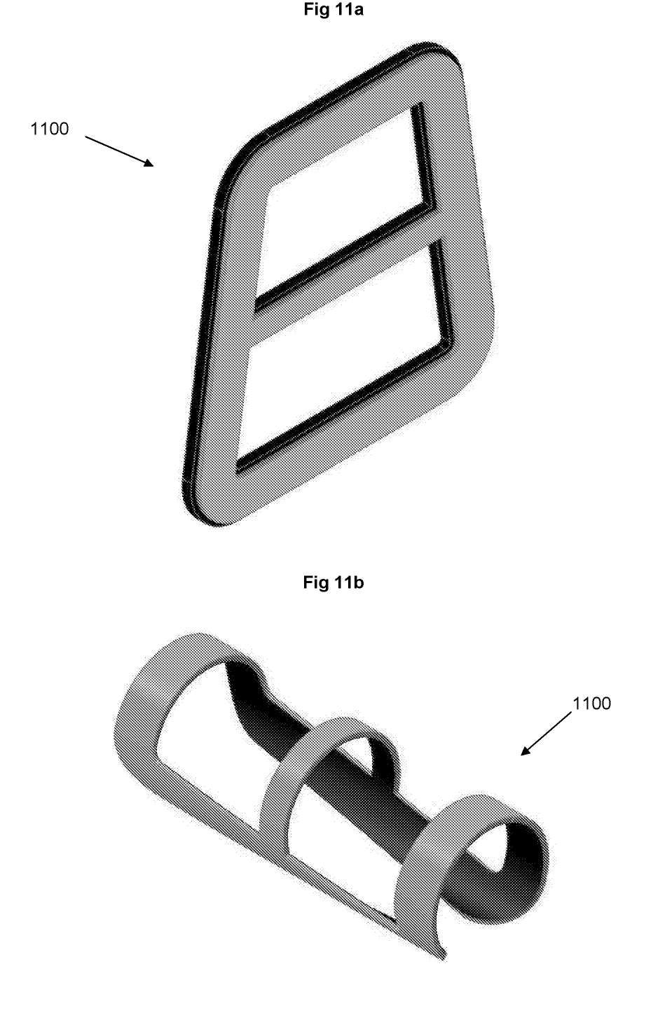

FIGS. 11a and 11b show perspective views of an eleventh embodiment of a splint according to the first aspect of the present invention.

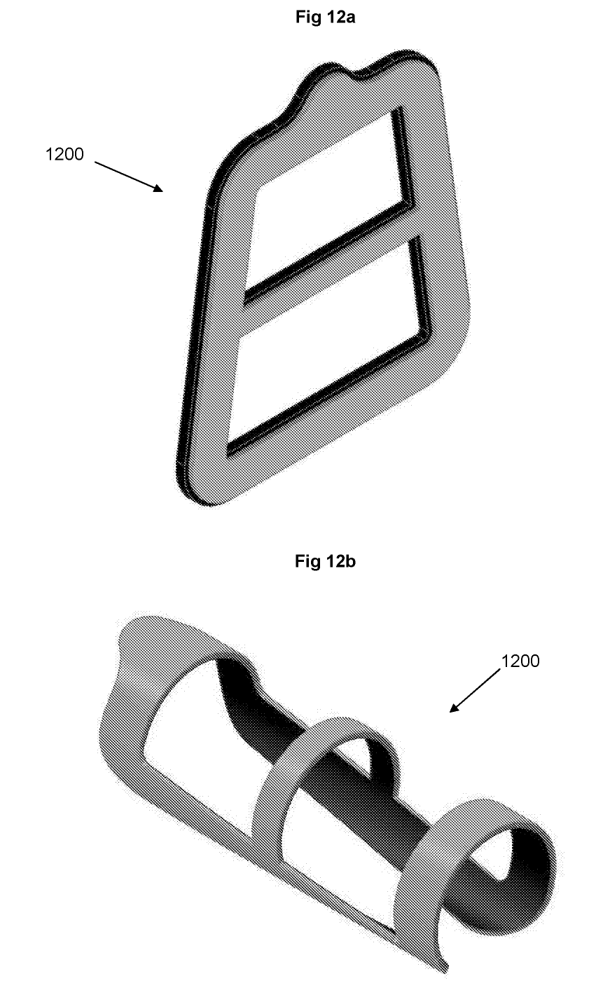

FIGS. 12a and 12b show perspective views of a twelfth embodiment of a splint according to the first aspect of the present invention.

FIG. 1a shows splint 100. Splint 100 has the form of a loop wherein the inner and outer edges of the loop are in the shape of a rounded square. As such, the inner edge of the loop defines a rounded square central aperture 102.

The loop of splint 100 can be considered to contain integrally formed first and second support portions 104 and 106, and first and second fixing portions 108 and 110.

Linear first and second support portions 104 and 106 are spaced apart by aperture 102. The spaced supports 104 and 106 are diametrically opposed and extend parallel to each other.

First and second fixing portions 108 and 110 are each formed of a curved section extending from either end of a linear section. Fixing portions 108 and 110 are spaced apart by aperture 102.

Fixing portion 108 is attached to support portions 104 and 106 at longitudinally opposed first ends of the support portions. Fixing portion 110 is attached to support portions 104 and 106 at longitudinally opposed second ends of the support portions. As such, fixing portions 108 and 110 extend outwardly from laterally opposite ends of each of support potions 104 and 106 so as to form a loop and provide structural rigidity to splint 100.

It will be appreciated that support portions 104 and 106 could serve as the fixing portions, and fixing portions 108 and 110 as the support portions. Further, either set of opposed rounded corners may serve as the first and second support portions 104 and 106.

The structure of splint 100 is a four layer laminate having base layer 112, core layer 114, decorative layer 116 (not shown) and top layer 118. Base layer 112 and decorative layer 116 are arranged on opposing faces of core layer 114. Top layer 118 is arranged on the upper face of decorative layer 116.

Base layer 112 is formed of resiliently deformable neoprene. Core layer 114 is formed of flexible aluminium. Top layer 118 is transparent plastic coating.

The splint 100 in FIG. 1a is shown in the open position wherein splint 100 has a planar conformation. FIG. 1b shows splint 100 in the closed position wherein splint 100 can be held around a digit.

The core 114 of splint 100 provides substantially all of the structural support for the splint. In the open position the depth of core layer 114 is about 1 mm and the width is about 7 mm. In this conformation core 114 is flexible such that it can easily be bent from the open planar position about the longitudinal X-X axis to the closed position, such as with the use of two fingers. It will be appreciated that if the support and fixing portions in splint 100 were swapped, then core 114 may also be bent from the open planar position to the closed position about a longitudinal Y-Y axis. However, due to the larger width the splint cannot easily be bent about the vertical Z-Z axis by the user.

Once in the closed position, the section of the core layer in the fixing portions has been rotated such that the depth of the core layer is now around 7 mm.

Accordingly, due to the increased depth it is now no longer easy for the user to bend the closed splint about the lateral axis. It will be appreciated that the presence of the digit within the split in the closed position prevents bending of the splint about the Z-Z axis.

In use, the user places splint 100 against the digit, for instance a finger, such that support portion 104 abuts the underneath face of the finger at a first point and support portion 106 abuts the underneath face of the finger at a second point distal to the first point. The user then applies pressure to the outer faces of fixing portions 108 and 110 such as to bend the inner face of the fixing portions about connections 118 to a position wherein fixing portions 108 and 110 abut opposing side faces of the finger. Fixing portions 108 and 110 may be further bent about line 120 to also partially abut the upper face of the finger. Pressure is applied to fixing portions 108 and 110 until the splint can be held around the finger without assistance.

Splint 100 may be bent into position using the pressure applied by a single finger to each of fixing portions 108 and 110.

When splint 100 is held around the finger it is in the closed position, as shown in FIG. 1b. In the closed position, fixing portions 108 and 110 hold splint 100 around the finger and also help to prevent lateral as well as extension movement of the finger beyond the metacarpophalangeal joint.

In the closed position, the relative positions of the first and second support portions 104 and 106 are maintained by the structural support provided by the increased depth of fixing portions 108 and 110. As such, flexion movement beyond the metacarpophalangeal joint is also restricted. Accordingly, in the closed position splint 100 immobilises the finger.

Splint 100 may be removed from the finger by simply pulling fixing portions 108 and 110 apart, for example by using one finger on each of the fixing portions.

FIGS. 2a and 2b show a splint 200 according to a second embodiment of the present invention. Splint 200 has the same structure as splint 100, except that splint 200 has an enlarged second support portion 202, which includes a planar tab projection extending coplanarly from the outer edge of the support portion.

As shown in FIG. 2b, splint 200 folds as for splint 100. In the closed position, the enlarged support portion 202 provides additional abutment with the underneath of the digit. The enlarged support portion may be example be used to restrict flexion of the distal inter-phalangeal joint.

FIGS. 3a and 3b show a splint 300 according to a third embodiment of the present invention. Splint 300 is the same as splint 100, except that splint 300 has a third supporting portion 302 extending across the centre of the aperture and which is connected at a first end to the midpoint of the inner edge of the first fixing portion and at a second end to the midpoint of the inner edge of the second fixing portion. As such, the fixing portions provide the linkages between the first, second and further supporting portions.

As shown in FIG. 3b, splint 300 folds as for splint 100. In the closed position, splint 300 provides three spaced parallel supporting portions that can abut the underneath face of the digit.

FIGS. 4a and 4b show a splint 400 according to a fourth embodiment of the present invention. Splint 400 is the same as splint 100, except that splint 400 contains the enlarged supporting portion of splint 200 and the third supporting portion of splint 300.

FIGS. 5a and 5b show a splint 500 according to a fifth embodiment of the present invention. Splint 500 is the same as splint 100, except that the inner and outer edges of splint 500 define a circle. As such, the loop of splint 500 is annular.

FIGS. 6a and 6b show a splint 600 according to a sixth embodiment of the present invention. Splint 600 is the same as splint 500, except that splint 500 contains the enlarged supporting portion of splint 200.

FIGS. 7a and 7b show a splint 700 according to a seventh embodiment of the present invention. Splint 700 is the same as splint 500, except that splint 500 contains the third supporting portion of splint 300.

FIGS. 8a and 8b show a splint 800 according to an eight embodiment of the present invention. Splint 800 is the same as splint 500, except that splint 800 contains the enlarged supporting portion of splint 200 and the third supporting portion of splint 300.

FIGS. 9a and 9b show a splint 900 according to a ninth embodiment of the present invention. Splint 900 is the same as splint 100, except that in splint 900 the end of the splint containing the first supporting portion 904 is wider than the end of the splint containing the second supporting portion 906.

As shown in FIG. 9b, splint 900 folds as for splint 500. However, in the closed position, the increased width of the end of the splint that contains the first support portion allows this end of splint 900 to accommodate a thicker part of the digit by still allowing the fixing portion to abut the upper face of the digit at a point close to the location of the first supporting portion.

FIGS. 10a and 10b show a splint 1000 according to an tenth embodiment of the present invention. Splint 1000 is the same as splint 900, except that splint 1000 contains the enlarged supporting portion of splint 200.

FIGS. 11a and 11b show a splint 1100 according to an eleventh embodiment of the present invention. Splint 1100 is the same as splint 900, except that splint 1100 contains the third supporting portion of splint 300.

FIGS. 12a and 12b show a splint 1200 according to a twelfth embodiment of the present invention. Splint 1200 is the same as splint 900, except that splint 1200 contains the enlarged supporting portion of splint 200 and the third supporting portion of splint 300.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A digit immobilizing splint comprising:
a first support portion configured to abut and support an underneath face of a digit on a first side of a joint of the digit while in use,
a second support portion configured to abut and support the underneath face of the digit on a second side of the joint while in use,
first and second fixing portions configured to respectively abut opposing side faces of the digit while in use,
wherein the first and second fixing portions each extend between the first support portion and the second support portion,
wherein the first and second fixing portions are configured to move between a first, open, position wherein the first and second fixing portions do not abut the digit when the first and second support portions are abutting the digit, and a second, closed, position wherein the first and second fixing portions abut opposing sides of the digit such as to immobilise the digit and hold the splint in position around the digit.

2. The splint according to claim 1, wherein the splint comprises one or more further support portions configured to abut a further point on the underneath face of the digit.

3. The splint according to claim 1, wherein the first and second fixing portions each extend between the first support portion and the second support portion to form a loop.

4. The splint according to claim 1, wherein one or more of the support portions comprise a tab.

5. The splint according to claim 1, wherein the fixing portions of the splint have a width that is greater than their depth when in the open position and a depth that is greater than their width when in the closed position.

6. The splint according to claim 1, wherein the fixing portions have a width of between 3 to 20 mm and a depth of 0.2 to 5 mm when in the open position.

7. The splint according to claim 1, wherein the splint has a total width in the open position of between 30 mm and 150 mm.

8. The splint according to claim 1, wherein splint has a first end that is wider than a second opposed end.

9. The splint according to claim 1, wherein the splint has a planar conformation in the open position.

10. The splint according to claim 1, wherein the splint is a laminate and further, comprises a flexible core layer and a padding layer operable to engage the surface of the digit.

11. (canceled)

12. A method of immobilizing a digit with with the splint of claim 1, the method comprising the steps of:
a. arranging the first and second support portions adjacent to the underside face of the digit;
b. moving the first and second fixing portions from the open position to the closed position wherein the fixing portions abut opposing sides of the digit immobilize to immobilize the digit and hold the split in place around the digit.

13. The method according to claim 12, further comprising moving the splint from the open position into the closed position by applying pressure to a single point on each side of the splint.
